(19) United States
(12) Patent Application Publication
(10) Pub. No.: US 2006/0019390 A1
(43) Pub. Date: Jan. 26, 2006

(54) Patterning substrate and cell culture substrate

(75) Inventors: Hideyuki Miyake, Tokyo (JP); Hideshi Hattori, Tokyo (JP); Hironori Kobayashi, Tokyo (JP)

Correspondence Address: SEYFARTH SHAW LLP, 55 EAST MONROE STREET, SUITE 4200, CHICAGO, IL, 60603-5803, US

(73) Assignee: Dai Nippon Printing Co., Ltd.

(21) Appl. No.: 11/044,978

(22) Filed: Jan. 27, 2005

(30) Foreign Application Priority Data
Jan. 28, 2004 (JP) 2004-020264

(51) Int. Cl. H01L 21/00; C12N 5/02

(52) U.S. Cl. 435/396; 438/1

(57) ABSTRACT

The present invention intends primarily to provide such as a cell culture patterning substrate that is used to adhere cells in a highly precise pattern on a base material to culture and a cell culture substrate on which cells is adhered in a highly precise pattern. To attain the above-mentioned object, the invention provides a patterning substrate, comprising a base material, a photocatalyst-containing layer which is formed on the base material and comprises at least a photocatalyst, and a cell adhesive layer which is formed on the photocatalyst-containing layer and at least comprises a cell adhesive material that has cell adhesive properties and is decomposed or denatured by action of the photocatalyst on the basis of irradiation with energy.

### BACKGROUND OF THE INVENTION

1. Field of the Invention

The present invention relates to a cell culture patterning substrate capable of adhering a cell in a highly precise pattern, a patterning substrate used for forming the cell culture patterning substrate, a coating liquid for patterning substrate used for forming the patterning substrate, and a cell culture substrate on which the cell is adhered in a highly precise pattern.

2. Description of the Related Art

At present, cell cultures of various animals and plants are performed, and also new cell culture methods are in development. The technologies of the cell culture are utilized, such as to elucidate the biochemical phenomena and natures of cells and to produce useful substances. Furthermore, with cultured cells, an attempt to investigate the physiological activity and toxicity of artificially synthesized medicals is under way.

Some cells, particularly a lot of animal cells have the adhesion dependency of adhering to some materials and growing thereon, and cannot survive for a long period under a flotation condition out of organisms. For culturing cells having such adhesion dependency, a carrier to which cells can adhere is necessary, and in general, a plastic culturing plate with uniformly applied cell adhesive proteins such as collagen, fibronectin and the like is used. It is known that these cell adhesive proteins act on cultured cells, make the cells adhere easily, and exert an influence on the form of cells.

On the other hand, there is a technology reported of adhering cultured cells only onto a small part on a base material and arranging them. By such a technology, it is made possible to apply cultured cells to artificial organs, biosensors, bioreactors and the like. As the method of arranging cultured cells, there is a method adopted in which a base material having a surface that forms a pattern different in easiness of adhesion to cells is used, cells are cultured on the surface of this base material and allowed to adhere only onto surfaces processed so that cells adhere, and thereby the cells are arranged.

For example, in Japanese Patent Application Laid-Open (JP-A) No. 2-245181, an electric charge-retaining medium on which an electrostatic pattern is formed is applied to culture cells for the purpose of proliferating nerve cells in a form of circuit, and the like. Furthermore, JP-A No. 3-7576 tries to arrange cultured cells on a surface on which a cell adhesion-inhibiting or cell adhesive photosensitive hydrophilic polymer has been patterned by a photolithography method.

Furthermore, JP-A No. 5-176753 discloses a cell culture base material on which a substance such as collagen and the like affecting on the adhesion ratio and form of cells is patterned, and a method of producing this base material by a photolithography method. By culturing cells on such a base material, a larger amount of cells can be adhered on a surface on which collagen or the like is patterned, to realize patterning of cells.

However, such patterning of cell culture regions may be required to be highly precise depending on applications. In the case of conducting patterning by such as a photolithography method using a photosensitive material as described above, a highly precise pattern can be obtained; however, a cell adhesive material is required to have photosensitivity, and it is difficult in many cases to conduct chemical modification to impart such photosensitivity to, for instance, biopolymers and the like; accordingly, there is a problem in that a range of selectivity of cell adhesive materials is extremely narrowed. Furthermore, in a photolithography method using a photo resist, it is necessary to use a liquid developer and the like, and these affect adversely in culturing cells in some cases.

Furthermore, as a method of forming a highly precise pattern of a cell adhesive material, a Micro Contact Printing method is proposed by George M. Whitesides, Harvard University (for example, U.S. Pat. Nos. 5,512,131 and 5,900,160 and JP-A Nos. 9-240125 and 10-12545). However, there is a problem in that it is difficult to industrially produce a cell culture base material having a pattern of a cell adhesive material using this method.

### SUMMARY OF THE INVENTION

In this connection, it is desired to provide a cell culture patterning substrate used to adhere and culture cells in a highly precise pattern on a base material, a cell culture substrate to which cells are adhered in a highly precise pattern, and the like.

The present invention provides a patterning substrate comprising a base material, a photocatalyst-containing layer which is formed on the base material and comprises at least a photocatalyst, and a cell adhesive layer which is formed on the photocatalyst-containing layer and at least comprises a cell adhesive material that has cell adhesive properties and is decomposed or denatured by action of the photocatalyst on a basis of irradiation with energy.

According to the present invention, the patterning substrate comprises the cell adhesive layer formed on the photocatalyst-containing layer; therefore, the irradiation of the patterning substrate with energy makes it possible to decompose or denature the cell adhesive material in the cell adhesive layer easily. According to this, the patterning substrate can be rendered a patterning substrate capable of forming easily a region having low adhesive properties to cells, wherein the cell adhesive material is decomposed or denatured, and a region having high adhesive properties to the cells, wherein the region is not irradiated with the energy, which comprises the cell adhesive material.

In the invention, a light-shielding portion may be formed in a pattern form on the base material or the photocatalyst-containing layer. According to this, only the cell adhesive material comprised in the cell adhesive layer on the region where the light-shielding portion is not formed can be decomposed or denatured by irradiating energy onto the patterning substrate from the side of the base material thereof. Thus, a region good in cell adhesive properties and a region poor in cell adhesive properties can easily be formed.

In the invention, it is preferred that the cell adhesive layer comprises therein a cell adhesion-inhibiting material having cell adhesion-inhibiting properties of inhibiting adhesion to cells at lease after the material is irradiated with energy. This makes it possible that cell adhesive properties of the region where the cell adhesive material is decomposed or denatured are made lower so as to cause cells to adhere, in a highly precise pattern, only onto the region where the cell adhesive material is decomposed or denatured.

The present invention also provides a cell culture patterning substrate, wherein the cell adhesive layer of any one of the above-mentioned patterning substrates comprises a cell adhesion-inhibiting portion where the cell adhesive material is decomposed or denatured in a pattern form, and a cell adhesion portion which is a region other than the cell adhesion-inhibiting portion.

According to the invention, the cell culture patterning substrate comprises the cell adhesion-inhibiting portion poor in cell adhesive properties, where the cell adhesive material is decomposed or denatured, and the cell adhesion portion good in cell adhesive properties, where the cell adhesive material is not decomposed or denatured; therefore, the cell culture patterning substrate can be rendered a cell culture patterning substrate capable of causing cells to adhere highly precisely only onto the cell adhesion portion without using any complicated step, any treating solution or the like that produces a bad effect on cells.

The present invention also provides a cell culture substrate, wherein cells adhere onto the cell adhesion portion of the above-mentioned cell culture patterning substrate.

According to the invention, the use of the cell culture patterning substrate having the above-mentioned cell adhesion portion and cell adhesion-inhibiting portion makes it possible that cells are caused to adhere easily onto the cell adhesion portion.

The present invention also provides a coating liquid for patterning substrate comprising a cell adhesive material which has cell adhesive properties and is decomposed or denatured by action of a photocatalyst on a basis of irradiation with energy, and a cell adhesion-inhibiting material which has cell adhesion-inhibiting properties of inhibiting adhesion to cells at least after the material is irradiated with energy.

According to the invention, when the coating liquid for patterning substrate is applied onto a layer or the like which contains a photocatalyst to form a layer, this layer is a layer good in cell adhesive properties through the above-mentioned cell adhesive material. Meanwhile, when this layer is irradiated with energy, the cell adhesive material can be decomposed or denatured; and further this layer comprises the above-mentioned cell adhesion-inhibiting material. For these reasons, the region irradiated with the energy can be rendered a region poor in cell adhesive properties. Accordingly, this coating liquid is a coating liquid for patterning substrate which is capable of forming easily a region good in cell adhesive properties and a region poor in cell adhesive properties.

The present invention also provides a method for producing a cell culture substrate comprising a base material, a photocatalyst-containing layer which is formed on the base material and comprises at least a photocatalyst, a cell adhesion portion which is formed in a pattern form on the photocatalyst-containing layer and at least comprises a cell adhesive material having cell adhesive properties, and a cell adhesion-inhibiting portion where the cell adhesive material is decomposed or denatured, cells adhering onto the cell adhesion portion, the method comprising: a cell adhesion process of causing the cells to adhere onto the cell adhesion portion; and a cell maintaining process of irradiating energy onto the cell adhesion-inhibiting portion, thereby maintaining the pattern of the cells adhering onto the cell adhesion portion.

According to the invention, owing to the cell adhesion process, after the cells are adhered on the cell adhesion portion, owing to the cell maintaining process, by irradiating energy on the cell adhesion-inhibiting portion on the cell culture substrate, the cells and the like adhered onto the cell adhesion-inhibiting portion can be removed, and thereby a cell culture substrate on which only on a cell adhesion portion cells are adhered in highly precise pattern can be prepared.

### BRIEF DESCRIPTION OF THE DRAWINGS

FIG. 1 is a schematic sectional view showing an example of the patterning substrate of the present invention.

FIG. 2 is a schematic sectional view showing another example of the patterning substrate of the invent-on.

FIG. 3 is a schematic sectional view showing still another example of the patterning substrate of the invention.

FIG. 4 is a schematic sectional view showing an example of the cell culture patterning substrate of the invention.

FIG. 5 is a schematic sectional view showing another example of the cell culture patterning substrate of the invention.

FIGS. 6A to 6C are process charts showing an example of a method for forming cell adhesion-inhibiting portions in the cell culture patterning substrate of the invention.

FIG. 7 is a schematic sectional view showing an example of the cell culture substrate of the invention.

FIG. 8 is a schematic sectional view showing an example of the energy irradiating step in the method of the invention for producing a cell culture substrate.

FIG. 9 is a schematic sectional view showing another example of the energy irradiating step in the method of the invention or producing a cell culture substrate.

FIGS. 10A and 10B are each a schematic sectional view for explaining a base material used in the cell culture patterning substrate of the invention.

### DESCRIPTION OF THE PREFERRED EMBODIMENTS

The present invention relates to such as a cell culture patterning substrate on which cells can be adhered in highly precise pattern, a patterning substrate that is used to form the cell culture patterning substrate, a coating liquid for patterning substrate that is used to form the patterning substrate, and a cell culture substrate on which cells are adhered in highly precise pattern. Hereinafter, these will be explained below separately.

A. Coating Liquid for Patterning Substrate

First, the coating liquid for patterning substrate of the present invention is described. This coating liquid for patterning substrate is a coating liquid comprising a cell adhesive material which has cell adhesive properties and is decomposed or denatured by action of a photocatalyst on the basis of irradiation with energy, and a cell adhesion-inhibiting material which has cell adhesion-inhibiting properties of inhibiting adhesion to cells at least after the material is irradiated with energy.

The coating liquid for patterning substrate of the invention comprises the above-mentioned cell adhesive material; therefore, in the case of applying the coating liquid for patterning substrate onto, for example, a layer or the like which comprises a photocatalyst to form a layer, the layer can be rendered a layer good in cell adhesive properties. In the case of irradiating the layer with energy, the cell adhesive material is decomposed or denatured by action of the photocatalyst on the basis of the irradiation with the energy so that the cell adhesive properties of the region irradiated with the energy can be made low. Since the coating liquid for patterning substrate comprises the above-mentioned cell adhesion-inhibiting material in this case, the cell adhesive properties of the region irradiated with the energy can be made lower by the cell adhesion-inhibiting properties of this cell adhesion-inhibiting material. Therefore, according to the invention, by using the coating liquid for patterning substrate to form a layer, cells can be caused to adhere, into the form of a highly precise pattern, only onto the region not irradiated with energy.

Each of the constituents used in the coating liquid for patterning substrate of the invention will be described hereinafter.

1. Cell Adhesive Material

First, a cell adhesive material used in the coating liquid for patterning substrate in the invention will be explained. The cell adhesive material used in the coating liquid for patterning substrate in the invention, as far as it has the cell adhesive properties and can be decomposed or denatured by action of a photocatalyst on the basis of irradiation with energy, is not particularly restricted to the kind and the like. Here, “having the cell adhesive properties” means being good in the cell adhesion and, for instance, when the cell adhesive properties is different depending on the kind of cells, means to be good in the adhesion with target cells.

The cell adhesive material used in the invention has such cell adhesive properties and can be decomposed or denatured by action of a photocatalyst on the basis of irradiation with energy such as to lose the cell adhesive properties or chance to one that has the cell adhesion-inhibiting properties that inhibit to adhere to cells.

As such materials having the cell adhesive properties, there are two kinds, one being materials having the cell adhesive properties owing to physicochemical characteristics and the other being materials having the cell adhesive properties owing to biochemical characteristics.

As physicochemical factors that determine the cell adhesive properties of materials having the cell adhesive properties owing to the physicochemical characteristics, the surface free energy, the electrostatic interaction and the like can be cited. For instance, in the case of the cell adhesive properties being determined by the surface free energy of the material, when the material has the surface free energy in a predetermined range, the adhesive properties between the cells and the material becomes good, and when it deviates from the above range the adhesive properties between the cells and material decreases. As such changes of the cell adhesive properties due to the surface free energy, experimental results such as shown in Data, for instance, CMC Publishing Co., Ltd. “Biomaterial no Saisentan”, Yoshito IKADA (editor), p. 109, lower part are known. As materials having the cell adhesive properties owing to such a factor, for instance, hydrophilic polystyrene, poly (N-isopropyl acrylamide) and the like can be cited. When such a material is used, by action of a photocatalyst on the basis of irradiation with energy, for instance, a functional group on a surface of the material is substituted, decomposed or the like to cause a change in the surface free energy, resulting in one that does not have the cell adhesive properties or one that has the cell adhesion-inhibiting properties.

When the adhesive properties between cells and a material is determined owing to the electrostatic interaction or the like, for instance, the cell adhesive properties can be determined owing to an amount of positive electric charges and the like that the material has. As materials having the cell adhesive properties owing to such electrostatic interaction, basic polymers such as polylysine, basic compounds such as aminopropyltriethoxysilane, N-(2-aminoethyl)-3-aminopropyltrimethoxysilane and condensates and the like including these can be cited. When such materials are used, by action of a photocatalyst on the basis of irradiation with energy, the above-mentioned materials are decomposed or denatured, thereby, for instance, an amount of positive electric charges present on a surface can be altered, resulting in one that does not have the cell adhesive properties or one that has the cell adhesion-inhibiting properties.

As materials having the cell adhesive properties owing to the biological characteristics, ones that are good in the adhesive properties with particular cells or ones that are good in the adhesive properties with many cells can be cited. Specifically, fibronectin, laminin, tenascin, vitronectin, RGD (arginine-glycine-asparagine acid) sequence containing peptide, YIGSR (tyrosine-isoleucine-glycine-serine-arginine) sequence containing peptide, collagen, atelocollagen, gelatin and the like can be cited. When such materials are used, by action of a photocatalyst on the basis of irradiation with energy, for instance, a structure of the material is partially destroyed, a principal chain is destroyed or the like, resulting in one that does not have the cell adhesive properties or one that has the cell adhesion-inhibiting properties.

Such a cell adhesive material, though different depending on the kind of the materials and the like, is contained in a coating liquid for patterning substrate normally in the range of 0.01 to 95% by weight, and preferably in the range of 1 to 10% by weight. Thereby, a region that contains the cell adhesive material can be made a region good in the cell adhesive properties.

2. Cell Adhesion-Inhibiting Material

The following will describe the cell adhesion-inhibiting material used in the coating liquid for patterning substrate of the invention. The cell adhesion-inhibiting material used in the coating liquid for patterning substrate of the invention is a material having cell adhesion-inhibiting properties of inhibiting adhesion to cells at least after the material is irradiated with energy. The cell adhesion-inhibiting properties mean natures that at the time of applying the coating liquid for patterning substrate to form a layer, cells are inhibited from adhering onto any region, in the layer, irradiated with energy. When adhesive properties to cells are different in accordance with the kinds of the cells, the cell adhesion-inhibiting properties mean natures that the layer has natures of inhibiting the layer from adhering onto target one out of the cells.

In the present invention, the cell adhesion-inhibiting material is not limited to any especial kind or the like if the material has such cell adhesion-inhibiting properties at least after the irradiation thereof with energy. The material may be, for example, a material having cell adhesion-inhibiting properties before the irradiation thereof with energy, a material which does not have cell adhesion-inhibiting properties before the irradiation thereof with energy but gives low cell adhesive properties by action of a photocatalyst on the basis of irradiation with energy to exhibit cell adhesion-inhibiting properties, or some other material.

The above-mentioned cell adhesion-inhibiting material having cell adhesion-inhibiting properties before the irradiation thereof with energy may be a material which exhibits cell adhesion-inhibiting properties without being decomposed or the like by action of a photocatalyst on the basis of irradiation with energy. Specific examples thereof include alkoxysilanes each having a fluoroalkyl chain and polycondension containing one or more out of the alkoxysilanes, amphoteric ion materials each having a betaine structure, phospholipid-containing materials, and poly(2-methoxyethyl) acrylate. In the case of using any one of such materials as the cell adhesion-inhibiting material, it is preferred that after the coating liquid for patterning substrate is applied to form a layer, the material is contained in the region which is not irradiated with energy, to such a degree that the cell adhesive properties of the above-mentioned cell adhesive material are not inhibited.

In the present invention, the cell adhesion-inhibiting material is preferably a material which does not have cell adhesion-inhibiting properties before the irradiation thereof with energy but gives low cell adhesive properties by action of a photocatalyst on the basis of irradiation with energy to exhibit cell adhesion-inhibiting properties for the following reason: at the time of applying the coating liquid for patterning substrate to form a layer in the case that such a material is used as the cell adhesion-inhibiting material, the cell adhesion-inhibiting material does not exhibit cell adhesion-inhibiting properties in the region, not irradiated with energy; therefore, the cell adhesion properties of the above-mentioned cell adhesive material are not hindered, and further in the region irradiated with energy the cell adhesive properties of a binder can be made lower.

The material used as such cell adhesion-inhibiting material is preferably, for example, a material which has such a high bonding energy that the main skeleton thereof is not decomposed by photoexcitation of the above-mentioned photocatalyst and which has an organic substituent as decomposed by action of the photocatalyst. Examples thereof include (1) organopolysiloxane which is obtained by hydrolyzing or polycondensating chloro- or alkoxy-silane or the like by such as sol-gel reaction and exhibits a large strength; and (2) organopolysiloxane which is obtained by crosslinking reactive silicone and is excellent in water repellency or oil repellency.

In the case of the (1), it is preferable to be organopolysiloxane that is a hydrolysis condensate or cohydrolysis condensate of at least one kind of silicon compounds expressed by a general formula:
YₙSiX₍₄₋ₙ₎
(Here, Y denotes an alkyl group, fluoroalkyl group, vinyl group, amino group, phenyl group or epoxy group, or organic groups including these, and X denotes an alkoxyl group, acetyl group or halogen. n is an integer of 0 to 3). The number of carbons of the group expressed with Y is preferably in the range of 1 to 20, and the alkoxy group expressed with X is preferably a methoxy group, ethoxy group, propoxy group or butoxy group.

As the reactive silicone according to the (2), compounds having a skeleton expressed by a general formula below can be cited.

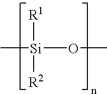

In the above general formula, n is an integer of 2 or more, R¹ and R² are each a substituted or unsubstituted alkyl, alkenyl, aryl or cyanoalkyl group having 1 to 20 of carbon atoms, and 40% or less by mole ratio of the whole thereof is composed of vinyl, phenyl and halogenated phenyl. A compound wherein R¹ and R² are each a methyl group is preferred since the surface energy thereof becomes the smallest; and it is preferred that 60% or more by mole ratio of the whole is methyl groups. The reactive silicone compound has, in the molecular chain thereof, at least one reactive group, such as a hydroxyl group, at a chain terminal or side chain thereof. The use of a material as described above makes it possible to render the surface of the region irradiated with energy a surface having a high hydrophilicity by action of the photocatalyst on the basis of the irradiation with the energy. According to this high hydrophilicity, it is possible to inhibit adhesion or cells so as to render the region irradiated with the energy a region onto which cells are not to adhere. It is allowable to incorporate, into a binder, a stable organosilicon compound which does not undergo any crosslinking reaction, such as dimethylpolysiloxane, together with the above-mentioned organopolysiloxane.

In the case of using the above-mentioned material as the cell adhesion-inhibiting material, the contact angle thereof with water is preferably from 15 to 120°, more preferably from 20 to 100° before the material is irradiated with energy. According to this, the cell adhesion-inhibiting material can be rendered a material which does not inhibit the cell adhesive properties of the above-mentioned cell adhesive material.

In the case of irradiating this cell adhesion-inhibiting material with energy, it is preferred that the contact angle thereof with water becomes 10° or less. This range makes it possible to render the material having a high hydrophilicity and low cell adhesive properties.

The contact angle with water referred to herein is a result obtained by using a contact angle measuring device (CA-Z model, manufactured by Kyowa Interface Science Co., Ltd.) to measure the contact angle of the material with water or a liquid having a contact angle equivalent to that of water (after 30 seconds from the time when droplets of the liquid are dropped down from its micro syringe), or a value obtained from a graph prepared from the result.

In the present invention, it is allowable to use, together with the above-mentioned cell adhesion-inhibiting material, a decomposition material or the like which causes a change in the wettability of the region irradiated with energy so as to make the cell adhesive properties thereof low, or which assists such a change.

As such decomposition substances, for instance, surfactants or the like that are decomposed and the like by action of a catalyst on the basis of irradiation with energy to be hydrophilic and the like to result in lowering the cell adhesive properties can be cited. Specifically, hydrocarbons of the respective series of NIKKO L BL, BC, BO, and BB manufactured by Nikko Chemicals Co., Ltd., and silicone base nonionic surfactants such as ZONYL FSN and FSO manufacture by Du Pont Kabushiki Kaisha, Surflon 5-141 and 145 manufactured by ASAHI GLASS CO., LTD., Megaface F-141 and 144 manufactured by DAINIPPON INK AND CHEMICALS, Inc., FTERGENT F-200 and F-251 manufactured by NEOS, UNIDYNEDS-401 and 402 manufactured by DAIKIN INDUSTRIES, Ltd., and Fluorad FC-170 and 176 manufactured by 3M can be cited, and cationic surfactants, anionic surfactants and amphoteric surfactants also can be used.

Other than the surfactants, oligomers and polymers such as polyvinyl alcohol, unsaturated polyester, acrylic resin, polyethylene, diallyl phthalate, ethylene propylene diene monomer, epoxy resin, phenol resin, polyurethane, melamine resin, polycarbonate, polyvinyl chloride, polyamide, polyimide, styrene-butadiene rubber, chloroprene rubber, polypropylene, polybutylene, polystyrene, polyvinyl acetate, nylon, polyester, polybutadiene, polybenzimidazole, polyacrylonitrile, epichlorohydrine, polysulfide, polyisoprene and the like can be cited.

In the present invention, such a cell adhesion-inhibiting material, though properly selected depending on the cell adhesion-inhibiting properties of the material used, is preferably contained, in the coating liquid for patterning substrate, in the range of 0.01 to 95% by weight, more preferably 0.1 to 10% by weight

3. Coating Liquid for Patterning Substrate

The following will describe the coating liquid for patterning substrate of the present invention. This coating liquid for patterning substrate is not limited to any special kind if the coating liquid contains the above-mentioned cell adhesive material and cell adhesion-inhibiting material. If necessary, the coating liquid may appropriately contain a binder and other components. The incorporation of the binder makes it possible to make easy the application of the coating liquid for patterning substrate onto, for example, a layer or the like which contains a photocatalyst and further give various properties, such as strength and resistance, to the formed layer. The binder may be appropriately selected in accordance with the use purpose of the coating liquid for patterning substrate. In the invention, the cell adhesion-inhibiting material may fulfill the role of this binder.

B. Patterning Substrate

The following will describe the patterning substrate of the present invention. This patterning substrate is a patterning substrate comprising a base material, a photocatalyst-containing layer which is formed on the base material and comprises at least a photocatalyst, and a cell adhesive layer which is formed on the photocatalyst-containing layer and at least comprises a cell adhesive material that has cell adhesive properties and is decomposed or denatured by action of the photocatalyst on the basis of irradiation with energy.

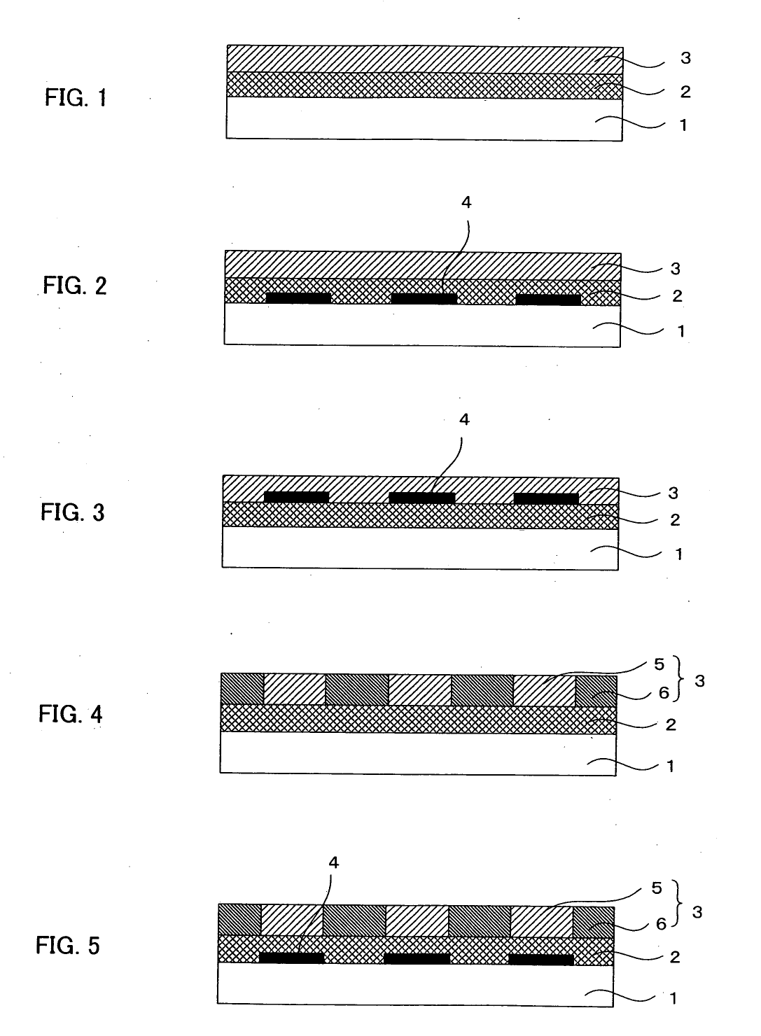

As shown in, for example, FIG. 1, the patterning substrate of the present invention is a substrate having a base material 1, a photocatalyst-containing layer 2 formed on the base material 1, and a cell adhesive layer 3 formed on the photocatalyst-containing layer 2.

According to the invention, the cell adhesive layer is formed on the photocatalyst-containing layer; therefore, the irradiation of the cell adhesive layer with energy makes it possible to decompose or denature easily the cell adhesive material contained in the cell adhesive layer by action of the photocatalyst in the adjacent photocatalyst-containing layer. According to this, the irradiation of energy in a pattern form makes it possible to form easily a region having low cell adhesive properties, wherein the cell adhesive material is decomposed or denatured, and a region having high cell adhesive properties wherein the region is not irradiated with the energy, where the cell adhesive material is not decomposed or denatured, without requiring any especial device or complicated step.

As shown in, for example, FIG. 2, the patterning substrate of the invention may be a substrate wherein light-shielding portions 4 are formed on the base material 1. As shown in, for example, FIG. 3, the patterning substrate may be a substrate wherein the light-shielding portions 4 are formed on the photocatalyst-containing layer 2 and the cell adhesive layer 3 is formed to cover this photocatalyst-containing layer 2 and the light-shielding portions 4. When such light-shielding portions are formed, only the cell adhesive material contained in the cell adhesive layer on the region where the light-shielding portions are not formed is decomposed or denatured when energy is irradiated onto the patterning substrate from the base material side or some other side. This makes it possible to radiate energy in a pattern form without using, for example, a photomask so as to form easily a region onto which cells are to adhere easily and a region onto which cells are not to adhere.

The following will describe each of the constituents of the patterning substrate of the invention.

1. Cell Adhesive Layer

First, the cell adhesive layer used in the patterning substrate of the invention is described. This cell adhesive layer is not limited to any especial kind if the layer is a layer which is formed on the photocatalyst-containing layer which will be described later as one of the constituents, and comprises at least a cell adhesive material which has cell adhesive properties and is decomposed or denatured by action of the photocatalyst on the basis of irradiation with energy.

The cell adhesive layer may be, for example, a layer formed by using a coating liquid containing a cell adhesive material which has cell adhesive properties and is decomposed or denatured by action of the photocatalyst on the basis of irradiation with energy. In the invention, the cell adhesive layer is in particular preferably a layer formed by using the above-mentioned coating liquid for patterning substrate, which comprises a cell adhesive material and a cell adhesion-inhibiting material for the following reason: this makes it possible that at the time of irradiating the cell adhesive layer with energy, the cell adhesive properties of the region irradiated with the energy are made lower so that cells are caused to adhere, in the form of a highly precise pattern, only onto the region not irradiated with the energy.

The method for forming such a cell adhesive layer may be a method of applying the above-mentioned coating liquid for patterning substrate in an ordinary coating manner or the like. Specifically, the coating manner which can be used may be spin coating, spray coating, dip coating, roll coating, bead coating or the like. An adsorbing process also can be preferably used.

When concave portions are formed in the base material which will be described later as one of the constituents, it is possible to use: a casting process of forming the photocatalyst-containing layer which will be described later as one of the constituents in the concave portions of the base material, dropping down the coating liquid for patterning substrate or the like into the concave portions, and drying the liquid so as to form the cell adhesive layer; an adsorbing process of forming a photocatalyst-containing layer, which will be described later, in the concave portions of the base material, dropping down the coating liquid for patterning substrate or the like into the concave portions, and washing the surface after a predetermined time; or some other process.

The cell adhesive material, the cell adhesion-inhibiting material and so on which are used in the cell adhesive layer used in the invention are the same as described in the above-mentioned item “A. Coating Liquid for Patterning Substrate”. Thus, the description thereof is omitted herein.

The film thickness of the cell adhesive layer is appropriately selected in accordance with the kind of the patterning substrate and others, and is usually from about 0.001 to 1 μm, preferably from about 0.005 to 0.1 μm.

2. Photocatalyst-Containg Layer

The following will describe the photocatalyst-containing layer used in the patterning substrate of the invention. This photocatalyst-containing layer is not limited to any especial kind if the layer is a layer comprising at least a photocatalyst, and may be a layer made only of a photocatalyst, or a layer which also comprises other components such as a binder.

The action mechanism of the photocatalyst, a typical example thereof being titanium oxide, which will be described later, in this photocatalyst-containing layer is not necessarily clear, but it has been considered as follows: carriers generated by the irradiation thereof with light react directly with a compound in the vicinity thereof, or are combined with active oxygen species generated in the presence of oxygen or water, hereby chancing the chemical structure of organic material. In the present invention, the carriers would act onto the cell adhesive material in the cell adhesive layer formed on the photocatalyst-contain ng layer.

As the photocatalyst that can be used in the present invention, specifically, for instance, titanium dioxide (TiO₂), zinc oxide (ZnO), tin oxide (SnO₂), strontium titanate (SrTiO₃), tungsten oxide (WO₃), bismuth oxide (Bi₂O₃) and iron oxide (Fe₂O₃) that are known as photo-semiconductors can be cited. These can be used singularly or in combination of at least two kinds.

In the present invention, in particular, titanium dioxide, owing to a large band gap, chemical stability, non-toxicity, and easy availability, can be preferably used. There are two types of titanium dioxide, anatase type and rutile type, and both can be used in the invention; however, the anatase type titanium dioxide is more preferable. An excitation wavelength of the anatase type titanium dioxide is 380 nm or less.

As such anatase type titanium dioxide, for instance, an anatase titania sol of hydrochloric acid deflocculation type (trade name: STS-02, manufactured by ISHIHARA SANGYO KAISYA, LTD., average particle diameter: 7 nm, and trade name: ST-KO1, manufactured by ISHIHARA SANGYO KAISYA, LTD.), an anatase titania sol of nitric acid deflocculation type (tradename: TA-15, manufactured by Nissan Chemical Industries Ltd., average particle diameter: 12 nm) and the like can be cited.

The smaller is a particle diameter of the photocatalyst, the better, because a photocatalyst reaction is caused more effectively. An average particle diameter of the photocatalyst is preferably 50 nm or less, and one having an average particle diameter of 20 nm or less can be particularly preferably used.

It is advantageous from the viewpoint of costs to use the photocatalyst-containing layer made only of a photocatalyst since the efficiency of decomposing or denaturing the cell adhesive material in the cell adhesive layer is improved to make the time for the treatment shorter. On the other hand, the use of the photocatalyst-containing layer made of a photocatalyst and a binder gives an advantage of making the formation of the photocatalyst-containing layer easy.

An example of the method for forming the photocatalyst-containing layer made only of a photocatalyst may be a vacuum film-forming method such as sputtering, CVD or vacuum vapor deposition. The formation of the photocatalyst-containing layer by the vacuum film-forming method makes it possible to render the layer a homogeneous photocatalyst-containing layer made only of a photocatalyst, thereby decomposing or denaturing the cell adhesive material homogeneously, and further thereby making the decomposition or denaturation of the cell adhesive material more effective in this case than in the case of using a binder also since the layer is made only of the photocatalyst. When concave portions are formed on the base material which will be described later as well, the photocatalyst-containing layer can be formed by the above-mentioned method.

Another example of the method for forming the photocatalyst-containing layer made only of a photocatalyst, is the following method: in the case that the photocatalyst is, for example, titanium dioxide, amorphous titania is formed on the base material and next fired so as to phase-change the titania to crystalline titania. The amorphous titania used in this case can be obtained, for example, by hydrolysis or dehydration condensation of an inorganic salt of titanium, such as titanium tetrachloride or titanium sulfate, or hydrolysis or dehydration condensation of an organic titanium compound, such as tetraethoxytitanium, tetraisopropoxytitanium, tetra-n-propoxytizanium, tetrabutoxytitanium or tetramethoxytitanium, in the presence of an acid. Next, the resultant is fired at 400 to 500° C. so as to be denatured to anatase type titania, and fired at 600 to 700° C. so as to be denatured to rutile type titania.

In the case of using a binder together with the above-mentioned photocatalyst, the binder is preferably a binder having a high bonding energy, wherein its main skeleton is not decomposed by photoexcitation of the photocatalyst. Examples of such a binder include the organopolysiloxanes described in the above-mentioned item “Cell Adhesive Layer”.

In the case of using such an organopolysiloxane as the binder, the photocatalyst-containing layer can be formed by dispersing a photocatalyst, the organopolysiloxane as the binder, and optional additives if needed into a solvent to prepare a coating liquid, and applying this coating liquid onto the base material which will be described later. The used solvent is preferably an alcoholic based organic solvent such as ethanol or isopropanol. The application can be performed by a known coating method such as spin coating, spray coating, dip coating, roll coating, or bead coating. When the coating liquid contains an ultraviolet curable component as the binder, the photocatalyst-containing layer can be formed by curing the coating liquid through the irradiation of ultraviolet rays onto the liquid.

When concave portions are formed on the base material which will be described later, it is allowable to perform such as a casting method of dropping down the above-mentioned coating liquid or the like into the concave portions and then drying the liquid to form the photocatalyst-containing layer.

As the binder, an amorphous silica precursor can be used. This amorphous silica precursor is preferably a silicon compound represented by the general formula SiX₄, wherein X are a halogen, a methoxy group, an ethoxy group, an acetyl group or the like; a silanol which is a hydrolyzate thereof; or a polysiloxane having an average molecular weight of 3000 or less.

Specific examples thereof include such as tetraethoxysilane, tetraisopropoxysilane, tetra-n-propoxysilane, tetrabutoxysilane, and tetramethoxysilane. In this case, the photocatalyst-containing layer can be formed by dispersing the amorphous silica precursor and particles of a photocatalyst homogeneously into a non-aqueous solvent, hydrolyzing the precursor with water content in the air to form a silanol onto a transparent base material, and then subjecting the silanol to dehydration polycondensation at room temperature. When the dehydration polycondensation of the silanol is performed at 100° C. or higher, the polymerization degree of the silanol increases so that the strength of the film surface can be improved. A single kind or two or more kinds of this binding agent may be used.

The content by percentage of the photocatalyst in the photocatalyst-containing layer is from 5 to 60% by weight, preferably from 20 to 40% by weight. The thickness of the photocatalyst-containing Layer is preferably from 0.05 to 10 μm.

Besides the above-mentioned photocatalyst and binder, the surfactant and so on described in the above-mentioned item “Cell Adhesive Layer” can be incorporated into the photocatalyst-containing layer.

In the present invention, it is preferred that the surface of the photocatalyst-containing layer is low in cell adhesive properties by having, for example, hydrophilicity for the following reason; this makes it possible that when the cell adhesive layer is decomposed and the like to make the photocatalyst-containing layer exposed, the exposed region is rendered a region low in cell adhesive properties.

In the invention, one or more light-shielding portions may be formed on the photocatalyst-containing layer, as described above. According to this, when the entire surface of the cell adhesive layer is irradiated with energy, the cell adhesive material contained in regions of the cell adhesive layer other than the regions thereof on which the light-shielding portions are formed can be decomposed or denatured without exciting the photocatalyst in the regions on which the light-shielding portions are formed. This case has an advantage that the direction in which the energy is irradiated is not particularly limited since the photocatalyst in the regions where light-shielding portions are formed is not excited.

Such light-shielding portions are not limited to any special kind if the portions make it possible to shield energy irradiated on the patterning substrate. The light-shielding portions may be formed, for example, by forming a thin film made of a metal such as chromium to have a thickness of about 1000 to 2000 Å by sputtering, vacuum vapor deposition or the like, and then patterning this thin film. The method for the patterning may be an ordinary patterning method such as sputtering.

The patterning method may be a method of forming, into a pattern form, a layer wherein shielding particles of carbon fine particles, a metal oxide, an inorganic pigment, an organic pigment or the like are incorporated into a resin binder. Examples of the used resin binder include a resin such as polyimide resin, acrylic resin, epoxy resin, polyacrylamide, polyvinyl alcohol, gelatin, casein or cellulose; mixtures made of two ore more thereof; photosensitive resins; and O/W emulsion type resin compositions, such as a material obtained by emulsifying a reactive silicone. The thickness of the light-shielding portions made of such a resin may be from 0.5 to 10 μm. The method for patterning the resin light-shielding portions may be an ordinarily-used method such as photolithography or printing.

3. Base Material

The following will describe the base material used in the present invention. This base material is not limited to any especial kind if the material is a material on which the photocatalyst-containing layer can be formed. For example, the base material may be made of an inorganic material such as metal, glass or silicon, or an organic material, a typical example of which is plastic.

The flexibility and the like of the base material are properly selected according to the kind of the patterning substrate, applications, or the like. Furthermore, the transparency of the base material is properly selected depending on such as the kind of the patterning substrate, or a direction in which energy that is irradiated to decompose or denature the cell adhesive material is irradiated. For instance, when the base material has such as the light-shielding portion and the energy is irradiated from a base material side and the like, the base material has the transparency.

In the invention, the base material may be a flat base material or a base material wherein one or more concave portions are formed. The base material may be a base material wherein a single concave portion is formed as shown in FIG. 10A, or a base material wherein plural concave portions are formed as shown in FIG. 10B.

At this time, side walls of the base material having the concave portion(s) may be treated in such a manner that the photocatalyst-containing layer or cell adhesive layer will not be formed thereon. Examples of the method for such a treatment include a method of using a mask or the like to cause a material having liquid repellency to adhere only onto the side walls by CVD; and a method of causing a material having liquid repellency to adhere onto the entire surface of the concave portion(s) and then using a cylindrical mask or the like to conduct ultraviolet ray treatment, plasma treatment or some other treatment, thereby making the bottom face(s) of the concave portion(s) lyophilic.

In the invention, the base material may be washed with a medical liquid, such as an alkali solution, and may be subjected to dry washing, such as oxygen plasma treatment or ultraviolet treatment. In this case, the wettability of the coating liquid or the like for forming the photocatalyst-containing layer is improved. Furthermore, this case has an advantage that the adhesive property of the base material to the photocatalyst-containing layer is improved since reactive functional groups are arranged on the surface of the base material.

As described above, one or more light-shielding portions may be formed on the base material of the invention. The light-shielding portions formed on the base material are equivalent to those described in the above-mentioned item “Photocatalyst-Containing Layer”. Thus, the description thereof is omitted herein. The light-shielding portions may be ones formed on the surface of the side at which the photocatalyst-containing layer is formed or ones formed on the opposite surface.

In the case of forming the light-shielding portions on the base material and forming the photocatalyst-containing layer on the light-shielding portions, a primer layer may be formed between the photocatalyst-containing layer and the light-shielding portions. The effect and function of this primer layer are not necessarily clear, but would be as follows: the primer layer exhibits a function of preventing the diffusion of impurities from openings which are present in the light-shielding portions and between the light-shielding portions, the impurities being factors for blocking the decomposition or denaturation of the cell adhesive material in the cell adhesive layer by action of the photocatalyst, in particular, residues generated when the light-shielding portions are patterned, or metal, metal ion impurities, or the like. Accordingly, the formation of the primer layer makes it possible to decompose or denature the cell adhesive material with high sensitivity so that a high resolution pattern can be obtained.

The primer layer in the present invention inhibits the impurities present not only in the light-shielding portion but also in the openings formed between the light-shielding portions from adversely affecting on an action of the photocatalyst; accordingly, the primer layer is preferably formed over an entire surface of the light-shielding portion including the openings.

The primer layer in the invention, as far as it is formed so that the light-shielding portion and the photocatalyst-containing layer may not be brought into contact, is not particularly restricted.

A material that forms the primer layer, though not particularly restricted, is preferably an inorganic material that is not likely to be decomposed by action of the photocatalyst. Specifically, amorphous silica can be cited. When such amorphous silica is used, a precursor of the amorphous silica is preferably a silicon compound that is represented by a general formula, SiX₄, X being halogen, methoxy group, ethoxy group, acetyl group or the like, silanol that is a hydrolysate thereof, or polysiloxane having an average molecular weight of 3000 or less.

A film thickness of the primer layer is preferably in the range of 0.001 to 1 μm and particularly preferably in the range of 0.001 to 0.1 μm.

4. Patterning Substrate

Subsequently, a patterning substrate in the invention will be explained. The patterning substrate in the invention, as far as it is one in which the above-mentioned photocatalyst-containing layer is formed on the base material and further the cell adhesive layer is formed on the photocatalyst-containing layer, is not particularly restricted. For instance, as needs arise, one in which another layer is further laminated and the like can be used.

C. Cell Culture Patterning Substrate

The following will describe the cell culture patterning substrate of the present invention. The cell culture patterning substrate is a patterning substrate wherein the cell adhesive layer of the above-mentioned patterning substrate comprises a cell adhesion-inhibiting portion where the cell adhesive material is decomposed or denatured in a pattern form, and a cell adhesion portion which is an a region other than the cell adhesion-inhibiting portion.

The cell culture patterning substrate of the invention is, for example, a substrate as shown in FIG. 4, which has a base material 1, a photocatalyst-containing layer 2 formed on the base material 1 and a cell adhesive layer 3 formed on the photocatalyst-containing layer 2, wherein the cell adhesive layer 3 has cell adhesion-inhibiting portions 6 where the cell adhesive material in the layer is decomposed or denatured, and cell adhesion portions 5 which are regions other than the cell adhesion-inhibiting portions 6, wherein the cell adhesive material is not decomposed or denatured.

According to the invention, the cell adhesion-inhibiting portions have low cell adhesive properties since the cell adhesive material therein is decomposed or denatured. The cell adhesion portions contain the cell adhesive material to exhibit high cell adhesive properties. Accordingly, cells can be caused to adhere only onto the cell adhesion portions without using any complicated step, any treating solution that produces a bad effect on the cells, or the like.

When cells are caused to adhere onto the cell adhesion portions and subsequently the entire surface thereof is irradiated with energy, the effect of the photocatalyst contained in the photocatalyst-containing layer makes it possible to decompose or denature the cell adhesive material in the cell adhesion portions to which the cells adhere, whereby the entire surface of the cell adhesive layer can be rendered a surface low in cell adhesive properties. This makes it possible to peel the cells adhering onto the cell adhesion portions to make the cells into a pattern form. The energy irradiated at this time is set so as to have such a degree that no effect is produced on the cells.

Here, the cell adhesion portion is a region that contains a cell adhesive material that has the cell adhesive properties and can be decomposed or denatured by action of the photocatalyst on the basis of irradiation with energy, and a region good in the adhesive properties with target cells. Here, having the cell adhesive properties means to have the physicochemical cell adhesive properties or the biological cell adhesive properties such as explained in the section of “A. Coating Liquid for Patterning Substrate”.

On the other hand, the cell adhesion-inhibiting portions are regions low in cell adhesive properties where the cell adhesive material is decomposed or denatured. The wording “the cell adhesive material is decomposed or denatured” herein means that the cell adhesive material is not contained or the cell adhesive material is contained in a smaller amount than the amount of the cell adhesive material contained in the cell adhesion portions. When the cell adhesive material is, for example, a material which can be decomposed by action of the photocatalyst on the basis of irradiation with energy, the wording means that a small amount of the cell adhesive material is contained in the cell adhesion-inhibiting portions; a decomposition product or the like of the cell adhesive material is contained; the cell adhesive layer is completely decomposed and removed so that the photocatalyst-containing layer is exposed; or the like. When the cell adhesive material is a material which can be denatured by action of the photocatalyst or the basis of irradiation with energy, the wording means that the denatured product or the like is contained in the cell adhesion-inhibiting portions. In the invention, it is preferred that the cell adhesion-inhibiting portions contain a cell adhesion-inhibiting material having cell adhesion-inhibiting properties such that adhesion to cells is inhibited. This makes it possible to make lower the cell adhesive properties of the cell adhesion-inhibiting portions so as to cause the cells to adhere highly precisely only onto the cell adhesion portions.

In the invention, light-shielding portions 4 may be formed, on the base material 1, in the same pattern form as the cell adhesion portions 5 of the cell adhesive layer 3 have, as shown in, for example, FIG. 5. The light-shielding portions may be formed on the photocatalyst-containing layer. When such light-shielding portions are formed, the cell adhesive material only on regions where no light-shielding portions are formed can be decomposed or denatured, for example, by forming a patterning substrate as described in the above-mentioned tem “B. Patterning Substrate” and then irradiating energy onto the entire surface thereof from the side of the substrate or the like. As a result, cell adhesion-inhibiting portions as described above can easily be formed.

In this case, energy can be irradiated only onto the cell adhesion-inhibiting portions by causing cells to adhere onto the cell adhesion portions of the cell culture patterning substrate of the invention and then irradiating energy onto the entire surface thereof from the side of its base material. This makes it possible that even in the case such as that cells adhere onto the cell adhesion-inhibiting portions, the cells on the cell adhesion-inhibiting portions can be removed by this energy irradiation, so as to keep a highly precise pattern.

The base material, the photocatalyst-containing layer and the cell adhesive layer used in the cell culture patterning substrate of the invention are equivalent to those described in the above-mentioned item “Patterning Substrate”. Thus, the description thereof is omitted herein. The following will describe the method for forming the cell adhesion-inhibiting portions.

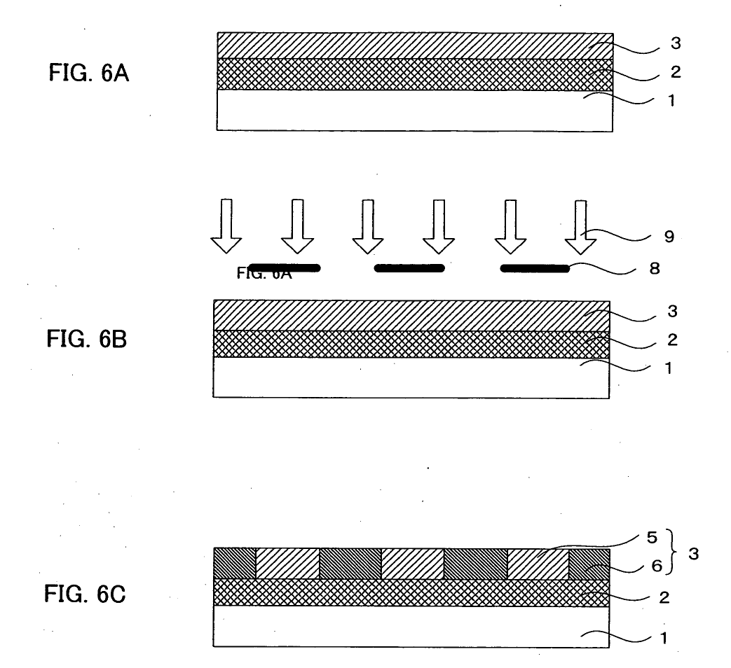

As shown in, for example, FIGS. 6A to 6C, a photocatalyst-containing layer 2 comprising at least a photocatalyst is first formed on a base material 1, and then a coating liquid for patterning substrate as described in the above-mentioned item “A. Coating Liquid for Patterning Substrate” or the like is used to form a cell adhesive layer 3 comprising a cell adhesive material (FIG. 6A). Next, for example, a photomask 8 is used to radiate energy 9, into the form of a pattern for forming cell adhesion-inhibiting portions, onto this cell adhesive layer 3 (FIG. 6B). According to this, the cell adhesive material in the cell adhesive layer 3 inside the regions irradiated with the energy is decomposed or denatured by action of the photocatalyst contained in the photocatalyst-containing layer 2, so that the regions irradiated with the energy and the regions not irradiated with the energy can be rendered cell adhesion-inhibiting portions 6 and cell adhesion portions 5, respectively (FIG. 6C). The methods for forming the cell adhesion portions and the photocatalyst-containing layer, and others are equivalent to those described in the above-mentioned item “B. Patterning Substrate”. Thus, the description thereof is omitted herein. As described above, it is preferred that the cell adhesion-inhibiting layer contains therein a cell adhesion-inhibiting material for inhibiting adhesion to cells at least after the layer is irradiated with energy. This makes it possible to make lower the cell adhesive properties of the cell adhesion-inhibiting portions.

The energy irradiation (exposure) mentioned in the present invention is a concept that includes all energy line irradiation that can decompose or denature the cell adhesive material by action of a photocatalyst on the basis of irradiation with energy, and is not restricted to light irradiation.

Normally, a wavelength of light used in such energy irradiation is set in the range of 400 nm or less, and preferably in the range of 380 nm or less. This is because, as mentioned above, the photocatalyst that is preferably used as a photocatalyst is titanium dioxide, and as energy that activates a photocatalyst action by the titanium oxide, light having the above-mentioned wavelength is preferable.

As a light source that can be used in such energy irradiation, a mercury lamp, metal halide lamp, xenon lamp, excimer lamp and other various kinds of light sources can be cited.

The method for the exposure may be a method of using a laser, such as an excimer laser or YAG laser, to draw and radiate energy in a pattern form, besides a method of using a light source as described above to draw and radiate energy in a pattern form through a photomask. When the base material has light-shielding portions in the same pattern form as the cell adhesion portions have, as described above, the exposure can be performed by irradiating the entire surface of the cell culture patterning substrate from the base material side thereof. When the photocatalyst-containing layer has thereon light-shielding portions in the same pattern form as the cell adhesion portions have, the exposure can be performed by irradiating the entire surface from any direction. These cases have an advantage that a photomask, and positioning and other steps are unnecessary.

When the base material has one or more concave portions and the photocatalyst-containing layer and the cell adhesive layer are formed in the concave portion(s), as described above, the exposure may be performed by any one of the above-mentioned methods. For instance, in the case of the plural concave portions, for example, the exposure may be performed into the form of patterns different from each other for the individual concave portions. Examples of the method for performing the exposure into the form of patterns different from each other for the individual concave portions as described above include a method of arranging different masks for the individual concave portions to radiate energy; and a method of arranging a chromium mask, a stencil mask or the like at the tip of an optical fiber to radiate energy.

In order not to radiate any energy onto side walls of the concave portions, the method for the exposure may be, for example, a method of using a cylindrical mask to radiate energy only onto the bottom faces of the concave portions.

The irradiation quantity of the energy at the time of the energy-irradiation is set to a value necessary for decomposing or denaturing the cell adhesive material by action of the photocatalyst.

In this case, it is preferred to irradiate the photocatalyst-containing layer with the energy while heating the layer since the sensitivity can be raised so as to decompose or denature the cell adhesive material effectively. Specifically, the layer is preferably heated at a temperature of 30 to 80° C.

The energy irradiation that is carried out through a photomask in the invention, when the above-mentioned base material is transparent, may be carried out from either direction of a base material side or a cell adhesive layer side. On the other hand, when the base material is opaque, it is necessary to apply energy irradiation from a cell adhesive layer side.

The cell culture patterning substrate of the invention is not limited to any especial kind if the substrate is a substrate wherein a photocatalyst-containing layer and a cell adhesive layer are formed on the base material, defined above, and further the cell adhesive layer has one or more cell adhesion portions and one or more cell adhesion-inhibiting portions. If necessary, one or more different layers may be appropriately formed in the patterning substrate.

In the invention, itis allowable to use, as the cell culture patterning substrate, such as a substrate obtained by forming the cell culture patterning substrate as described above, cutting one portion from the patterning substrate, and attaching this portion onto the bottom of a base material in a concave form, or the like.

D. Cell Culture Substrate

Next, a cell culture substrate in the invention will be explained. A cell culture substrate in the invention is one in which cells are adhered onto the cell adhesion portion in the above-mentioned cell culture patterning substrate.

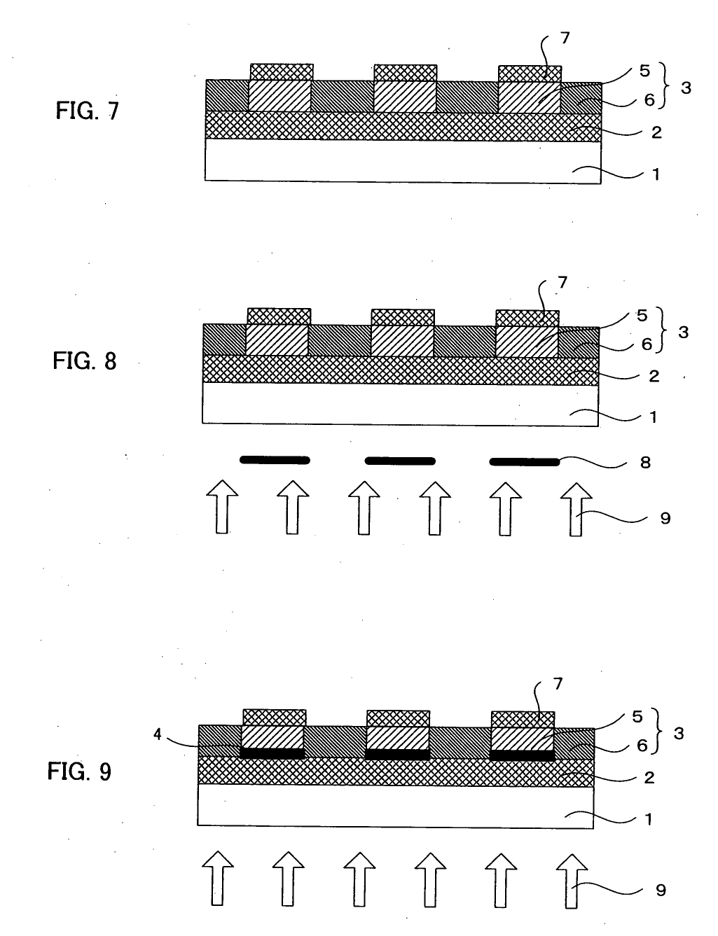

In the cell culture substrate in the invention, as shown in, for example, FIG. 7, cells 7 are adhered only onto a cell adhesion portion 5 of the cell adhesive layer 3, and, on a cell adhesion-inhibiting portion 6, cells 7 are not adhered.

In the invention, on the cell culture patterning substrate, a cell adhesion portion good in the cell adhesive properties and a cell adhesion-inhibiting portion that does not have the cell adhesive properties are formed. Accordingly, for instance, even when cells are coated over an entire surface of the cell culture patterning substrate, the cells can be adhered to the cell adhesion portion only and the cells on the cell adhesion-inhibiting portion can be readily removed. Thereby, without using any complicated step, any treating solution that produces a bad effect on the cells, or the like, the cell culture substrate can be readily formed.

According to the invention, by irradiating energy onto the entire surface of the cell culture substrate, the cell adhesive material in the cell adhesion portions to which cells adhere can be decomposed or denatured to make low the cell adhesive properties of the entire surface of the cell adhesive layer. This makes it possible to peel the cells adhering onto the cell adhesion portions easily to yield only cells formed in a pattern form. The energy irradiated at this time is set in to such a degree that the energy produces no effect on the cells.

As mentioned above, in the case of a light-shielding portion being formed on a base material in a pattern same as to the cell adhesion-inhibiting portion, when energy is irradiated over an entire surface from the base material side as needs arise, cells adhered onto a cell adhesion-inhibiting portion can be removed; accordingly, a pattern in which cells are adhered to the cell adhesion portion can be maintained in high precision.

In the case of the above-mentioned light-shielding portion being not formed, when energy is irradiated by using a photomask or the like in which an opening is formed in a pattern same as to the cell adhesion-inhibiting portion, a highly precise pattern can be maintained.

Hereinafter, cells that are used in a cell culture substrate in the invention will be explained. Since an explanation of a cell culture patterning substrate is same as that in the “C. Cell Culture Patterning Substrate”, here it is omitted.

(Cells)

As cells used in a cell culture substrate in the invention, as far as cells can adhere onto a cell adhesion portion of the cell culture patterning substrate but do not adhere to a cell adhesion-inhibiting portion, there is no particular restriction.

As cells used in the present invention, except for, for instance, non-adhesive cells such as nervous tissue, liver, kidney, pancreas, blood vessel, brain, cartilage and blood corpuscle, all tissues present in an organism and cells derived therefrom can be used. Furthermore, since even for generally non-adhesive cells, recently, in order to adhere and fix, a technology of modifying a cell membrane is devised; accordingly, as needs arise, the non-adhesive cells, when this technology is applied, can be used in the present invention.

The respective tissues such as mentioned above are formed of cells having various functions; accordingly, it is necessary to select desired cells to use. For instance, in the case of the liver, it is formed of, other than hepatocytes, epithelial cells, endothelial cells, Kupffer's cells, fibroblasts, and fat-storing cells and the like. In this case, since the adhesive properties with a cell adhesive material is different depending on the kinds of the cells, in accordance with a cell strain, a cell adhesive material used in the cell adhesion portion and a composition ratio thereof have to be selected.

A method of adhering cells to the cell adhesion portion, as far as it can adhere cells only on the cell adhesion portion of the cell culture patterning substrate that has the cell adhesion portion and the cell adhesion-inhibiting portion, is not particularly restricted. For instance, cells may be adhered by use of an ink jet printer, a manipulator or the like; however, a method in which after a cell suspension is disseminated to adhere cells on the cell adhesion portion, unnecessary cells on a cell adhesion-inhibiting portion are washed with a phosphate buffer to remove the cells is generally used. As such a method, a method described in, for instance, a reference literature, Kevin E. Healy et al., “Spatial distribution of mammalian cells dicated by material surface chemistry”, Biotech. Bioeng. (1994), p. 792 can be used,

E. Method for Producing a Cell Culture Substrate

The following will describe the method of the present invention for producing a cell culture substrate. This method is a method for producing a cell culture substrate comprising a base material, a photocatalyst-containing layer which is formed on the base material and comprises at least a photocatalyst, a cell adhesion portion which is formed in a pattern form on the photocatalyst-containing layer and at least comprises a cell adhesive material having cell adhesive properties, and a cell adhesion-inhibiting portion where the cell adhesive material is decomposed or denatured, cells adhering onto the cell adhesion portion, the method comprising: a cell maintaining process for irradiating energy on the cell adhesion-inhibiting portion to maintain a pattern of the cell adhered onto the cell adhesion portion, after a cell adhesion process for adhering cells on the cell adhesion portion is carried out.

As shown in, for example, FIG. 8, the method of the invention for producing a cell culture substrate is a method for producing a cell culture substrate having a base material 1, a photocatalyst-containing layer 2 formed on the base material 1, a cell adhesive layer 3 which is formed on the photocatalyst-containing layer 2 ard has cell adhesion portions 5 and cell adhesion-inhibiting portions 6, and cells 7 formed on the cell adhesion portions 5. The method of the invention comprises a cell adhesion process of causing the cells 7 to adhere onto the cell adhesion portions 5; and a subsequent cell maintaining process of irradiating energy 9 through a photomask 8 or the like, onto the cell adhesion-inhibiting portions 6, thereby maintaining the pattern of the cells 7 adhering onto the cell adhesion portions 5. This makes it possible to remove the cells on the cell adhesion-inhibiting portions by action of the photocatalyst contained in the photocatalyst-containing layer even if the cells adhere onto the cell adhesion-inhibiting portions. Consequently, a cell culture substrate wherein the cells adhere highly precisely only onto the cell adhesion portions can be prepared.

The following will describe the energy irradiating process in the method of the invention for producing a cell culture substrate.

(Cell Maintaining Process)

The cell maintaining process in the method of manufacturing a cell culture substrate in the invention is the process in which after the cell adhesion process for adhering cells onto the cell adhesion portion is carried out, energy is irradiated on the cell adhesion-inhibiting portion to maintain a pattern of cells adhered on the cell adhesion portion, and as far as it can allow maintaining a pattern of cells on the cell adhesion portion by irradiating energy on the cell adhesion-inhibiting portion, there is no particular restriction on a method of irradiating energy and the like.

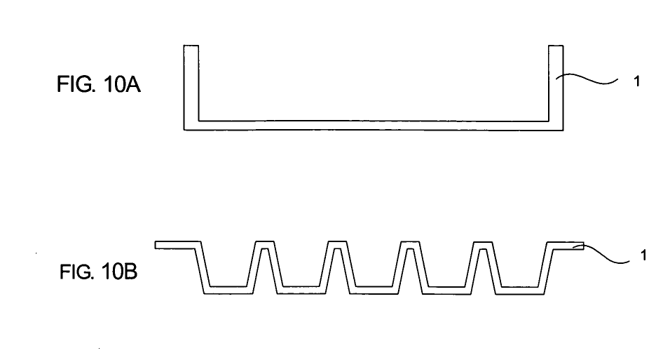

In the present invention, as shown in, for example, FIG. 8, a method and the like in which by using photomask 8 and the like having an opening in a pattern same as to the cell adhesion-inhibiting portion energy 9 is irradiated can be cited. Furthermore, as shown in, for example, FIG. 9, when a light-shielding portion 4 on a base material 1 is formed in a pattern same as to the cell adhesion portion 5, a method in which energy is irradiated over an entire surface from the base material 1 side to irradiate energy 9 only on the cell adhesion-inhibiting portion 6 can be used.

At that time, energy that is irradiated, as far as it can remove the cells adhered on the cell adhesion-inhibiting portion by action of the photocatalyst on the basis of irradiation with energy, is not particularly restricted. Specifically, it can be same as that explained in the section of the cell culture pattern layer of the “° C. Cell Culture Patterning Substrate”; accordingly, explanation thereof is not repeated here.

As for a timing when the process is applied, it may be carried out immediately after the adhering cells where cells are adhered on the cell adhesion portion is carried out, or when cells are cultured for a predetermined period on the cell adhesion portion, in order to avoid inconveniences such as that the cells adhere on the cell adhesion-inhibiting portion to result in a wide pattern and the like, in accordance with a kind and state of the cells, the timing may be properly selected. The process, also, may be repeated.

The cell adhesion process for adhering cells into the cell adhesion portion is same as that of the method of adhering cells explained in the section of the “D. Cell Culture Substrate”, therefore, an explanation here is omitted.

(Others)

Besides the above-mentioned energy irradiating process and cell adhesion process, the method of the invention for producing a cell culture substrate may comprise, for example, the process of forming a photocatalyst-containing layer on the base material, the process of forming a cell adhesive layer on the photocatalyst-containing layer, the process of irradiating patterned energy onto the cell adhesive layer to form a cell adhesion portion and a cell adhesion-inhibiting portion, and other optional processes if necessary.

A photocatalyst, cell adhesive material, base material, cells and the like that are used in the method of manufacturing a cell culture substrate in the invention and a method of forming the cell adhesion-inhibiting portion are same as that described in the section of the “D. Cell Culture Substrate”, explanations thereof are not repeated here.

The present invention is not limited to the above-mentioned embodiments. The embodiments are mere examples. All modifications which have substantially the same structure as the technical conception described in the claims of the invention and produce effects and advantages similar to those of the technical conception are included in the technical scope of the invention.

### EXAMPLES

Hereinafter, examples and comparative examples are shown and thereby the present invention will be more specifically described.

### Example 1

(Formation of a Photocatalyst-Containing Layer)

Three grams of isopropyl alcohol, 0.4 g of an organosilane, TSL 8114 (manufactured by GE Toshiba Silicones), and 1.5 g of a photocatalyst inorganic coating agent, ST-K01 (manufactured by ISEIHARA SANGYO KAISYA, LTD.) were mixed, and then the mixture was heated at 100° C. for 20 minutes while stirred.

This solution was applied onto a glass substrate subjected to alkali treatment in advance, 0.7 mm in thickness, by spin coating method, and the substrate was dried at 150° C. for 10 minutes to advance hydrolysis and polycondensation reaction, thereby forming, on the substrate, a photocatalyst-containing layer, 0.2 μm in thickness, wherein the photocatalyst was strongly fixed into an organopolysiloxane.

(Formation of a Cell Adhesive Layer)

An aqueous solution wherein 0.2 mg of a Fibronectin F-4759 (manufactured by Sigma) and 200 ml of pure water were mixed and was dropped down onto the photocatalyst-containing layer of the above-mentioned substrate at a rate of 300 μl per cm² of the area of the substrate, and this was allowed to stand still at 4° C. for 24 hours. Furthermore, the substrate was washed with PBS two times, and then exposed to nitrogen gas so as to be dried, thereby yielding a patterning substrate having, on the glass substrate thereof, the photocatalyst-containing layer and a cell adhesive layer.

(Patterning of the Patterning Substrate)

Subsequently, ultraviolet rays were irradiated from a mercury lamp, through a stripe-form photomask having light-shielding portions of 80 μm width and space portions of 300 μm width, onto this patterning substrate at 5 J/cm² (wavelength: 254 nm) to yield a cell culture patterning substrate having the cell adhesion layer patterned in such a manner that the unexposed portions had cell adhesive properties and the exposed portions had cell adhesion-inhibiting properties.

(Adhesion of Cells)

About the process of experiments for culturing cells originating from various kinds of tissues, details thereof are described in, for example, “Soshikibaiyo no Gijyutsu, Dai San Han, Kiso”, edited by The Japanese Tissue Culture Association and published by Asakura Shoten, and other documents. In the present application, rat hepatocytes were used to evaluate the cell culture patterning substrate.

A liver extracted from a rat was transferred into a Petri dish, and the liver was cut into pieces 5 mm in size with a scalpel. Thereto was added 20 ml of a DMEM culture medium, and the pieces were lightly suspended with a pipette. Thereafter, the suspension was filtrated with a cell filter. The resultant cell-coarsely-dispersed suspension was subjected to centrifugation at 500 to 600 rpm for 90 seconds, and the supernatant was sucked to be removed. A new DMEM culture medium was added to the remaining cells, and the resultant was again subjected to centrifugation. This operation was repeated three times to yield substantially homogenous hepatocytes. To the resultant hepatocytes was added 20 ml of a DMEM culture medium, and the cells were suspended therein to prepare a hepatocytes suspension. Next, 900 ml of distilled water was added to 14.12 g of a Waymouth MB 752/1 culture medium (containing L-glutamine but containing no NaHCO₃) (manufactured by GIBCO). To this were added 2.24 g of NaHCO₃, 10 ml of an amphotericin B solution (ICN), and 10 ml of a penicillin streptomycin solution (manufactured by GIBCO), and this solution was stirred. This was adjusted into a pH of 7.4, and then the total amount thereof was set to 1000 ml. The resultant was filtrated with a 0.22 μm membrane filter and sterilized to prepare a Waymouth MB752/1 culture medium solution. The previously-prepared hepatocytes suspension was suspended into the prepared Waymouth MB 752/1 culture medium solution, and further the resultant suspension was inoculated onto the above-mentioned cell culture patterning substrate, which had the cell adhesion portions and the cell adhesion-inhibiting portions. This substrate was allowed to stand still in an incubator to which 5% CO₂ was supplied at 37° C. for 24 hours to cause the hepatocytes to adhere onto the entire surface of the substrate. This substrate was washed with PBS two times to remove non-adhering cells and dead cells. Thereafter, the culture medium solution was exchanged for a new culture medium solution. While the exchange of the culture medium solution was repeated, the cells were continued to be cultured for 48 hours. The cells were then observed with an optical microscope. As a result, it was found out that the cells adhered along the cell adhesion portions of the cell culture patterning substrate.

### Example 2

(Formation of a Cell Culture Patterning Substrate)

A quartz substrate, wherein a stripe-form shielding layer having light-shielding portions of 80 μm width and space portions of 300 μm width was formed on a surface of a base material, was prepared. A photocatalyst-containing layer and a cell adhesive layer were formed on the surface of this substrate with the shielding layer in the same way as in Example 1. Next, instead of using any photomask, ultraviolet rays were irradiated onto the entire back face of the substrate from the back face side of the substrate under the same conditions as in Example 1 to form a cell culture patterning substrate.

(Adhesion of Cells)

Cells were caused to adhere onto the above-mentioned patterning substrate in the same way as in Example 1. As a result, in the present example also, it was found out that the cells adhered along the cell adhesion portions of the cell culture patterning substrate.

### Example 3

(Formation of a Photocatalyst-Containing Layer)

A photocatalyst-containing layer was formed on a glass substrate in the same way as in Example 1.

(Formation of a Layer Containing a Cell Adhesive Material)

Five (5.0) grams of an organosilane TSL8114 (manufactured by GE Toshiba Silicones), 0.55 g of an alkylsilane, TSL 8241 (manufactured by GE Toshiba Silicone) and 2.36 g of 0.005-N hydrochloric acid were mixed, and the components were mixed for 12 hours while stirred. This solution was applied onto a quartz substrate subjected to alkali treatment in advance by spin coating, and the substrate was dried at 150° C. for 10 minutes to form, on the substrate, a material layer the properties of which were changeable cell adhesive properties to cell adhesion-inhibiting properties by decomposition reaction based on a photocatalyst. In this way, a patterning substrate was prepared.

(Patterning of the Patterning Substrate)

Ultraviolet rays were irradiated from a mercury lamp (wavelength; 254 nm), through a stripe-form photomask having light-shielding portions of 80 μm width and space portions of 300 μm width, onto this substrate at 5 J/cm² to yield a cell culture patterning substrate having a cell adhesive surface patterned in such a manner that the unexposed portions had cell adhesive properties and the exposed portions had cell adhesion-inhibiting properties.

(Adhesion of Cells)

Cells were caused to adhere onto the above-mentioned patterning substrate in the same way as in Example 1. As a result, in the present example also, it was found out that the cells adhered along the cell adhesion portions of the cell culture patterning substrate.

### Example 4

A hole of 14 mm diameter was made at the center of the bottom face of a commercially available plastic dish (manufactured by Corning Inc.) of 35 mm diameter. Subsequently, a glass substrate of about 0.1 mm thickness was used to form a cell culture patterning substrate in the same way as in Example 1. This cell culture patterning substrate was cut into a 21 mm square. Thereafter, the glass substrate of the cut cell culture patterning substrate was stuck onto the above-mentioned plastic dish through an adhesive agent, KE45T (Shin-Etsu Chemical Co., Ltd).

(Adhesion of Cells)

The plastic dish was sterilized with 70% ethanol, and washed with PBS. Thereafter, the dish was washed with a DMEM culture medium. Cells were then cultured in the same way as in Example 1. As a result, it was found out that the cells adhered along the cell adhesion portions in the plastic dish.

### Example 5

A quartz substrate with a shielding layer, about 0.1 mm in thickness, was used to form a cell culture patterning substrate having a cell adhesive layer in the same way as in Example 2. This cell culture patterning substrate was cut into a 21 mm square. Thereafter, in the same way as in Example 4, the quartz substrate of the cut cell culture patterning substrate was stuck onto the above-mentioned plastic dish.

(Adhesion of Cells)

The plastic dish was sterilized with ultraviolet rays, and subsequently cells were cultured in the plastic dish in the same way as in Example 4. As a result, it was found out that the cells adhered along the cell adhesion portions in the plastic dish.

### Example 6

A quartz substrate with a shielding layer, about 0.1 mm in thickness, was used to form a cell culture patterning substrate having a cell adhesive layer in the same way as in Example 3. This cell culture patterning substrate was cut into a 21 mm square. Thereafter, in the same way as in Example 4, the quartz substrate of the cut cell culture patterning substrate was stuck onto the above-mentioned plastic dish.

(Adhesion of Cells)

Cells were cultured in the plastic dish in the same way as in Example 4. As a result, it was found out that that the cells adhered along the cell adhesion portions in the plastic dish.

### Example 7

(Formation of a Patterning Substrate)

A photocatalyst-containing layer was formed on a substrate in the same way as in Example 1. Subsequently, a soluble type I collagen solution (type I-C, manufactured by Nitta Gelatin Inc.) was diluted 10 times with a solution of hydrochloric acid aqueous solution, the pH of which was adjusted into 3. This was applied onto the photocatalyst-containing layer by spin coating, so as to form a patterning substrate.

(Patterning of the Patterning Substrate, and Adhesion of Cells)

The patterning substrate was patterned in the same way as in Example 1, so as to form a cell culture patterning substrate. Thereafter, cells were caused to adhere onto the cell culture patterning substrate in the same way as in Example 1. As a result, in the present examples also, it was found out that the cells adhered along the cell adhesion portions on the cell culture patterning substrate.

What is claimed is:

1. A patterning substrate, comprising a base material, a photocatalyst-containing layer which is formed on the base material and comprises at least a photocatalyst, and a cell adhesive layer which is formed on the photocatalyst-containing layer and at least comprises a cell adhesive material that has cell adhesive properties and is decomposed or denatured by action of the photocatalyst on a basis of irradiation with energy.

2. The patterning substrate according to claim 1, wherein a light-shielding portion is formed in a pattern form on the base material or the photocatalyst-containing layer.

3. The patterning substrate according to claim 1, wherein the cell adhesive layer comprises therein a cell adhesion-inhibiting material having cell adhesion-inhibiting properties of inhibiting adhesion to cells at least after the material is irradiated with energy.

4. The patterning substrate according to claim 2, wherein the cell adhesive layer comprises therein a cell adhesion-inhibiting material having cell adhesion-inhibiting properties of inhibiting adhesion to cells at least after the material is irradiated with energy.

5. A cell culture patterning substrate, wherein the cell adhesive layer of the patterning substrate according to claim 1 comprises a cell adhesion-inhibiting portion where the cell adhesive material is decomposed or denatured in a pattern form, and a cell adhesion portion which is a region other than the cell adhesion-inhibiting portion.

6. A cell culture patterning substrate, wherein the cell adhesive layer of the patterning substrate according to claim 2 comprises a cell adhesion-inhibiting portion where the cell adhesive material is decomposed or denatured in the pattern form, and a cell adhesion portion which is a region other than the cell adhesion-inhibiting portion.

7. A cell culture patterning substrate, wherein the cell adhesive layer of the patterning substrate according to claim 3 comprises a cell adhesion-inhibiting portion where the cell adhesive material is decomposed or denatured in a pattern form, and a cell adhesion portion which is a region other than the cell adhesion-inhibiting portion.

8. A cell culture patterning substrate, wherein the cell adhesive layer of the patterning substrate according to claim 4 comprises a cell adhesion-inhibiting portion where the cell adhesive material is decomposed or denatured in the pattern form, and a cell adhesion portion which is a region other than the cell adhesion-inhibiting portion.

9. A cell culture substrate, wherein cells adhere onto the cell adhesion portion of the cell culture patterning substrate according to claim 5.

10. A cell culture substrate, wherein cells adhere onto the cell adhesion portion of the cell culture patterning substrate according to claim 6.

11. A cell culture substrate, wherein the cells adhere on-o the cell adhesion portion of the cell culture patterning substrate according to claim 7.

12. A cell culture substrate, wherein the cells adhere onto the cell adhesion portion of the cell culture patterning substrate according to claim 8.

13. A coating liquid for patterning substrate, comprising a cell adhesive material which has cell adhesive properties and is decomposed or denatured by action of a photocatalyst on a basis of irradiation with energy, and a cell adhesion-inhibiting material which has cell adhesion-inhibiting properties of inhibiting adhesion to cells at least after the material is irradiated with energy.

14. A method for producing a cell culture substrate comprising a base material, a photocatalyst-containing layer which is formed on the base material and comprises at least a photocatalyst, a cell adhesion portion which is formed in a pattern form on the photocatalyst-containing layer and comprises at least a cell adhesive material having cell adhesive properties, and a cell adhesion-inhibiting portion where the cell adhesive material is decomposed or denatured, cells adhering onto the cell adhesion portion, the method comprising: a cell adhesion process of causing the cells to adhere onto the cell adhesion portion; and a cell maintaining process of irradiating energy onto the cell adhesion-inhibiting portion, thereby maintaining the pattern of the cells adhering onto the cell adhesion portion.
